(19) United States
(12) Patent Application Publication
(10) Pub. No.: US 2005/0250674 A1
(43) Pub. Date: Nov. 10, 2005

(54) Nutritional and food supplement preparation comprising creatine and orotic acid complexes, derivatives and analogs

(76) Inventor: Daniel Amato, Wellington, FL (US)

Correspondence Address: John P. Luther, Jenkens & Gilchrist, 225 W. Washington Street, Suite 2600, Chicago, IL, 60606, US

(21) Appl. No.: 10/971,501

(22) Filed: Oct. 21, 2004

(51) Int. Cl. A61K 38/16; A01N 37/44; A01N 37/12; A61K 31/195; A61K 38/00; A01N 37/18

(52) U.S. Cl. 514/2

(57) ABSTRACT

The present invention provides nutritional, pharmaceutical, or dietetic preparations that comprise creatine and orotic acid derivatives, creatine-orotic acid complexes and derivatives thereof, and functional analogs of such compounds and compositions, and the use of these compounds and compositions in the treatment of enhanced body performance of humans and animals, or in the prevention of specific diseases that are related to nutritional disorders.

### FIELD OF THE INVENTION

This invention relates to nutritional, pharmaceutical, or dietetic preparations that comprise creatine and orotic acid derivatives, creatine-orotic acid complexes and derivatives thereof, and functional analogs of such compounds and compositions and the use of these compounds and compositions in the treatment of enhanced body performance of humans and animals or in the prevention of specific diseases that are related to nutritional disorders.

### BACKGROUND OF THE INVENTION

Copyright 2004 Daniel Amato. All rights reserved. A portion of the disclosure of this patent application/patent document contains material which is subject to copyright protection. The copyright owner has no objection to the facsimile reproduction by anyone of the patent document or the patent disclosure as it appears in the United States Patent and Trademark Office file or records.

Creatine, or methyl guanidine-acetic acid, as known, is biosynthesized in humans, and is oftentimes taken as a dietary supplement. This compound is manifested in the liver through transamidation and transmethylation of arginine, glycine and methionine, and up to about 98% is stored in muscle mass, with the remaining amount stored in various other parts of the body.

The creatine molecule is metabolized in the body into phosphocreatine by creatine kinase which in turn is metabolized into creatinine. Phosphocreatne is a high energy molecule which provides a rapid supply of adenosine tri-phosphate (ATP) to muscles, with the creatinine metabolite product ultimately being released into the blood and excreted by the kidney as a metabolic waste, or as a by product of skeletal muscle creatine metabolism. As the amounts of ATP in muscle mass are relatively small, phosphocreatine provides for a high concentration of ATP in muscles during periods of muscle exertion. This biosynthetic reaction pathway may be generally shown as follows:

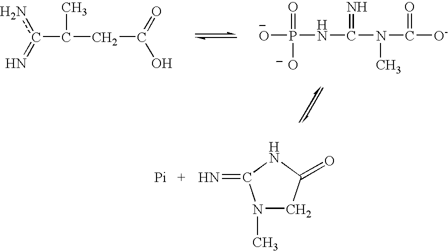

Therefore, creatine is known to play an important role in the regulation and homeostasis of skeletal muscle metabolism. It is correspondingly available in many food sources, such as the skeletal muscles of animals and fish. However, as many high concentration creatine-containing foods are also high in fat and cholesterol it has become advisable to use creatine supplements as a source of this important nutrient. In general, the reported oral ingestion of creatine of from about 20 to 30 g per day, divided between several doses, for several days can lead to a greater than 20% increase in human skeletal muscle total creatine content. See, for example, International Patent Application Publication W094/0212 disclosing that the administration of creatine in amounts of from 15% per day (0.2 to 0.48/kg body weight per day) for two days will increase muscular strength. Others report that maintenance doses of creatine to accumulate and remain in sufficient stores in muscle mass range from about 4 to 12 g per day. See, for example, U.S. Pat. No. 5,773,473.

An entire industry has thus developed for food supplements containing creatine, typically creatine monohydrate, as commonly used by athletes to extend endurance, to allow for increased training, and to provide for enhanced muscle size and strength. Creatine as a strength and endurance enhancer has become particularly attractive as an alternative and substitute for oftentimes problem plagued steroids and steroidal compositions and preparations, with many creatine-containing products now available in the marketplace.

In one example, EP0669 083 describes a creatine beverage product and its preparation which is said to provide creatine in stable form without readily converting to biologically inactive creatinine.

U.S. Pat. No. 6,274,161 describes compositions containing creatine in suspension in an edible supporting matrix or foodstuff, such as dairy or cheese spreads, caviar, meat spreads and the like.

In U.S. Pat. No. 6,114,379 there are disclosed various bio-available chelates of creatine bonded to essential minerals. These chelate compounds are described as capable of being absorbed in the stomach or intestines via active transport without substantial metabolism of the chelate complex.

U.S. Pat. No. 6,620,425 discloses a variety of food supplements and methods comprising creatine and lipoic acid. These supplements are said to be particularly adapted for supplementing the diet of an athlete, and the increase of an athlete's muscle size and/or strength while avoiding the use of steroids.

Further, U.S. patent application Publication No. 2003/001/3767 describes a method of employing a creatine compound to treat muscle loss associated with liver and kidney diseases.

Additionally U.S. Pat. No. 6,172,114 discloses a creatine supplement which comprises creatine and ribose in a pharmaceutically acceptable vehicle for internal administration, and which may include such additional nutrients as vitamins, minerals, amino acids and liquid carbohydrates.

Metabolic supplementation with orotic acid and orotate derivatives, such as magnesium orotate, has also been reported to enhance exercise tolerance in athletes, and also in patients with coronary heart disease. See, Cordiovasc Drugs Ther. September 1998; 12 Suppl. 2: 147-52. Orotic acid is a natural substance found in humans and also in various foods including diary products. Orotates are also known as a component of a natural system of electrolyte carriers for distributing minerals throughout the body. Hieper, Re-calcification of bone metastases by calcium diorotate, Agressologic, 1970: 11(6): 495-502.

Orotates are oftentimes described as being those components that after consumption will provide orotate ions in the blood plasma. Some examples of orotate ion include, without limitation, orotic acid (6-carboxy-2,4-dihydroxypyrimidine), salts thereof, such as sodium or potassium, or zinc salts, esters such as choline, or methyl esters, and extracts which are rich in orotic acid, such as certain extracts from the liver. Precursors, such as argenine, glutamine or aspartate are also contemplated.

Generally, orotate is known to ensure proper biosynthesis of pyrimidines, and to neutralize any excess of ribose that may be formed in some conditions, and to also ensure sufficient levels of beta-alanine, camosine and anserine. Some daily doses of orotate are thought to comprise 0.1 to 8 g and when administered as a precursor, in excess of 5 grams is generally preferred. See, for example, U.S. patent application Publication No. 20020183263.

Acyl derivatives of orotate have been reported useful as pyrimidine neucleotide precursors in the prophylactic and therapeutic treatment of humans and animals to enhance resistance to bacterial endotoxin and other inflammatory stimuli and inflammatory mediators. See, for example, U.S. patent application Publication 2004033981. Uridine, cytidine and orotate have also been studied as to their effects on liver function in hepatic disorders.

Additionally, in International Application No. PCT/FR 82/00159, acylated derivatives of dehydro orotic acid and preparations thereof are reported as useful antihypotensive pharmaceutical compositions. Such acyl derivatives include N-acyl dehydro orotic acids in which one of the nitrogen atoms is acylated by a propionic acid derivative.

Accordingly, it would be beneficial to provide nutritional and food supplement formulations and preparations, comprising creatine and/or orotic acid/orotate derivatives and complexes thereof for enhanced bio-availability of these important body performance enhancing substances.

### SUMMARY OF THE INVENTION

The present invention provides novel nutritional and food supplement preparations comprising creatine and oratic acid derivatives, creatine and oratic acid complexes and derivatives and analogs thereof.

The invention is more fully explained and understood with reference to the following Detailed Discussion of Preferred Embodiments.

DETAILED DISCUSSION OF PREFERRED EMBODIMENTS

All patent references, published patent applications and literature references referred to or cited herein are expressly incorporated by reference to the same extent as if each were specifically and individually indicated to be incorporated by reference. Any inconsistency between these publications and the present disclosure is intended to and shall be resolved in favor of the present disclosure.

Throughout this specification and the claims which follow, unless the context requires otherwise, the word “comprise” and variations such as “comprises” and “comprising” will be understood to imply the inclusion of a stated compound, bio-active agent, carrier, vehicle,-adjuvant or excipient or other material or group thereof, but not the exclusion of any other compound, bio-active agent, carrier, vehicle, adjuvant, or excipient or other material or group thereof.

The language “in combination with” another agent includes co-administration of the compound or compounds of the invention (“compound(s)”) and the agent, administration of the compound(s) of the invention first, followed by other agent and administration of other agent first, followed by the compound(s) of the invention.

In accordance with the present invention there is provided novel nutritional and food supplement preparations useful for enhancing physical performance and endurance in mammals, especially human athletes, which comprise one or more compounds selected from the following formulae: 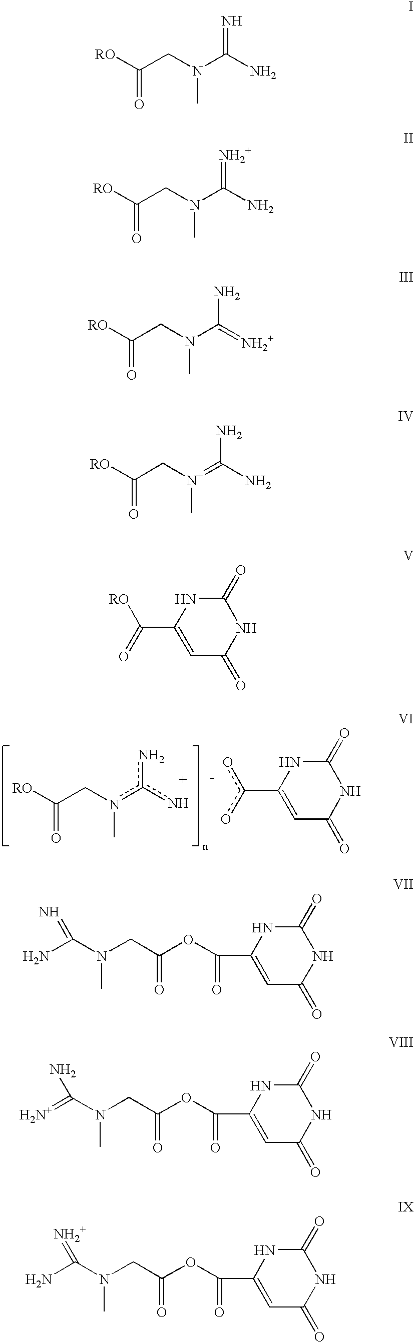 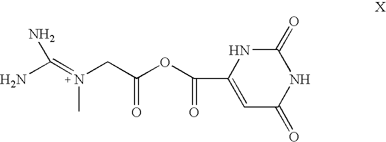
and in which R is a residue of a biologically significant amino acid, or naturally occurring amino acid, selected from arginine, glycine, alanine, beta-alanine, valine, isovaline, norvaline, leucine, isoleucine, norleucine, alloisoleucine, phenylalanine, proline, serine, homoserine, allothreonine, threonine, tyrosine, cysteine, cystine, homocysteine, methionine, lysine, histidine, tryptophan, aspartic acid, glutamic acid, asparagine, glutamine, taurine, sarcosine, ornithine, citrulline, aminobutyric acid, aminoisobutyric acid, amino-n-butyric acid, pyroglutamic acid, thiaproline, seleno-L-methionine, hydroxyproline, 4-hydroxyproline, 5-hydroxyproline, aminoadipic acid, cadaverine (15-diaminopontane), 4-amino-benzoic acid, x-aminopimelic acid, 2,4-diamino-n-butyric acid, gylcine-glycine, glycine-proline, threonine-aspartic acid, hydroxylysine, diaminopimelic acid, proline-hydroxyproline, lysine-alanine, dopamine, cystathionine, 3,4-dihydroxy-phenylalanine, argene-succinic acid, gama-amino butyric acid, statine, carnitine, ethionine, serotonin, peptides, oligo-peptides and proteins thereof, and pharmaceutically salts thereof, or

R is one or more one or more of hydrogen, hydroxy, halogen, alkyl, cycloalkyl, substituted alkyl, substituted cycloalkyl, heterocylclic, substituted heterocyclic, alkenyl, substituted alkenyl, alkynl, substituted alkynyl, aryl, substituted aryl, heteroaryl, substituted hereroaryl, alkylaryl, substituted alkylaryl, arylalkyl, substituted arylalkenyl, substituted arylalkenyl, arylalkynl, substituted arylalkynl, aroyl, substituted aroyl, acyl, substituted acyl, or the like and any and all isomers and totomeric forms thereof, or the two R groups can cooperate to form a 5-, 6- or 7-membered ring including N and the two R groups, or either of the R groups is a divalent moiety selected from the group consisting of alkylene, substituted alkylene, oxyalklene, substituted oxyalkylene, alkenylene, substituted alkenylene, arylene, substituted arylene, alkarylene, substituted alkarylene, aralkylene and substituted aralkylene, wherein said divalent moiety serves as the same substituent for two dithiocarbamate structures, thereby linking said structures together so as to form a bis(dithiocarbamate) species; and n is an integer from 1 to 3.

As used herein the term “biologically significant amino acid” refers to any amino acid known in the art to be bioactive in mammals to any degree, or which displays any amount of efficacy for any indication in humans and animals.

The term “oligo-peptides” as used herein refer to small chain amino acid combinations, such as, for instance, synthetic growth hormone releasing peptides (GHRPs), of which an example is ghrelin, a 28 amino acid octanoylated peptide.

The term “alkyl” as used herein refers to straight chain, branched and cyclic fully saturated hydrocarbon residues, preferably straight chain or branched alkyl. Some non-limiting examples useful herein of straight and branched alkyl moieties of the invention include C 1-20 alkyl, such as methyl, ethyl, propyl, isopropyl, butyl, isobutyl, see-butyl, tert-butyl, amvl, isoamyl, sec-amyl, I, 2-dimethylpropyl, 1,1-dimethylpropyl, hexyl, 4-methylpentyl, 1 methylpentyl, 2-methylpentyl, 3-methylpentyl, 1,1-demethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1,methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,2,2-trimethylbutyl, 1,1,3-trimethylbutyl, octyl, 6-methylheptyl, 1 methylheptyl, 1,1,3,3-tetramethylbutyl, nonyl, 1,2,3,4,5,6, or 7-methyloctyl, 1,2,3,4, or 5-ethylheptyl, 1,2, or 3-propylhexyl, decyl, 1,2,3,4,5,6,7 and 8-methylnonyl, 1,2,3,4,5, or 6-ethyloctyl, 1,2,3, or 4-propyllheptyl, undecyl, 1,2,3,4,5,6,7,8 or 9-methyldecyl, 1,2,3,4,5,6, or 7-ethylnonyl, 1,2,3,4, or 5-propyloctyl, 1,2, or 3-butylheptyl, 1-pentylhexyl, dodecyl, 1,2,3,4,5,6,7,8,9, or 10-methylundecyl, 1,2,3,4,5,6,7, or 8-ethyldecyl, 1,2,3,4,5, or 6-propylnonyl, 1,2,3, or 4-butyloctyl or 1,2-pentylheptyl and the like.

Additional alkyl examples include C 21-25 alkyl, C 26-30 alkyl, C 31-35 alkyl, C 36-40 alkyl, C 41-46 alkyl, C 47-55 alkyl, C 56-60 alkyl, with examples of cyclic alkyls including mono- and polycyclic alkyl groups, such as cyclopropyl, cyclobutyl, cyclohexyl, cycloheptyl, cyclooctyl, cyclononyl, cyclodecyl and the like.

Further, as used herein, the term “alkenyl” refers to groups formed from straight chain, branched or cyclic hydrocarbon residues containing at least one carbon-carbon double bond, including ethylenically mono-, di-, or polyunsaturated alkyl or cycloalkyl groups as exemplified and defined above. Some representative examples of alkenyl groups or moieties include C 1-20 alkenyl, such as vinyl, allyl, 1-methylvinyl, butenyl, iso-butenyl, 3-methyl-2-butenyl, 1-pentenyl, cyclopentyl, 1-methylcyclopentyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, and 1,3,5,7-cyclooctatetraenyl.

Additional examples of alkenyl groups include C 10-15 alkenyl, C 16-20 alkenyl, C 21-25 alkenyl, C 26-30 alkenyl, C 31-35 alkenyl, C 36-40 alkenyl, C 41-45 alkenyl, C 46-50 alkenyl, C 51-55 alkenyl, C 56-60 alkenyl, C 61-65 alkenyl, C 66-70 alkenyl, and C 71-80 alkenyl, with each of these examples possibly containing one or more alkyl or alkyl branches.

As employed herein, “substituted alkyl” comprises alkyl groups further bearing one or more substituents selected from hydroxyl, alkoxy (of a lower alkyl group), mercapto (of a lower alkyl group), cycloalkyl, substituted cycloalkyl, heteroaryl, aryloxy, substituted aryloxy, halogen, trifluoromethyl, cyano, nitro, nitrone, amino, amido, —C(O)H, acyl, oxyacyl, carboxyl, carbamate, sulfonyl, sulfonamide, sulfuryl, and the like.

As employed herein, “cycloalkyl” refers to cyclic ring-containing groups containing in the range of about 3 up to 8 carbon atoms, and “substituted cycloalkyl” refers to cycloalkyl groups further bearing one or more substituents as set forth above.

As employed herein, “heterocyclic” refers to cyclic (i.e., ring-containing) groups containing one or more heteroatoms (e.g., N, O, S, or the like) as part of the ring structure, and having in the range of 3 up to 14 carbon atoms and “substituted heterocyclic” refers to heterocyclic groups further bearing one or more substituents as set forth above.

As employed herein, “alkenyl” refers to straight or branched chain hydrocarbyl groups having a least one carbon—carbon double bond, and having in the range of about 2 up to 12 carbon atoms, and “substituted alkenyl” refers to alkenyl groups further bearing one or more substituents as set forth above.

As employed herein, “alkynyl” refers to straight or branched chain hydrocarbyl groups having at least one carbon—carbon triple bond, and having in the range of about 2 up to 12 carbon atoms, and “substituted alkynyl” refers to alkynyl groups further bearing one or more substituents as set forth above.

As employed herein, “aryl” refers to aromatic groups having in the range of 6 up to 14 carbon atoms and “substituted aryl” refers to aryl groups further bearing one or more substituents as set forth above.

As employed herein, “heteroaryl” refers to aromatic groups containing one or more heteroatoms (e.g., N, O, S, or the like) as part of the ring structure, and having in the range of 3 up to 14 carbon atoms and “substituted heteroaryl” refers to heteroaryl groups further bearing one or more substituents as set forth above.

As employed herein, “alkylaryl” refers to alkyl-substituted aryl groups and “substituted alkylaryl” refers to alkylaryl groups further bearing one or more substituents as set forth above.

As employed herein, arylalkyl” refers to aryl-substituted alkyl groups and “substituted arylalkyl” refers to arylalkyl groups further bearing one or more substituents as set forth above.

As employed herein, “arylalkenyl” refers to aryl-substituted alkenyl groups and “substituted arylalkenyl” refers to arylalkenyl groups further bearing one or more substituents as set forth above.

As employed herein, “arylalkynyll” refers to aryl-substituted alkynyl groups and “substituted arylalkynyl” refers to arylalkynyl groups further bearing one or more substituents as set forth above.

As employed herein, “aroyl” refers to aryl-carbonyl species such as benzoyl and “substituted aroyl” refers to aroyl groups further bearing one or more substituents as set forth above.

As employed herein, “acyl” refers to alkyl-carbonyl species.

As employed herein, “halogen” refers to fluoride, chloride, bromide or iodide atoms.

Further, amino acids used herein to form ester analogs useful in the nutrient compositions of the invention may be natural, non-natural, or of natural extract origin, or a racemate, or an optically active material.

Some non-limiting examples of additional esters which may be used herein include methyl formate, propyl formate, isobutyl formate, butyl formate, methyl acetate, ethyl acetate, propyl acetate, isopropyl acetate, sec-butyl acetate, isobutyl acetate, butyl acetate, amyl acetate, sec-amyl acetate, iso-amyl acetate, sec-hexyl acetate, hexyl acetate, heptyl acetate, octyl acetate, methyl propionate, methyl butyrate, isobutyl isobutyrate, butyl butyrate, methyl methanoate, ethyl methanoate, butyl methanoate, methyl ethanoate, ethyl ethanoate, propyl ethanoate, 2-propyl ethanoate, butyl ethanoate, dimethyl ethyl ethanoate, methylpropyl ethanoate, pentyl ethanoate, 2 pentyl ethanoate, 3-methylbutyl ethanoate, hexyl ethanoate, 2-heptyl ethanoate, octyl ethanoate, 2-ethylhexyl ethanoate, nonyl ethanoate, methyl propanoate, ethyl propanoate, propyl propanoate, butyl propanoate, dimethyl ethyl propanoate, 3-methylbutyl propanoate, hexyl propanoate, heptyl propanoate alkyl undeconoate, carbonate, and poly-carbonate esters.

Some particularly preferred compounds of the invention include, for example, arginine orotate, diarginine orotate, citrulline orotate, and momo, di-, and tri-creatine orotates and their esterified derivatives.

Preparation of the creatine and orotic esters of the invention, or of the inventive esterified creatine-orotate complexes, may be accomplished by any conventional method, of which several are known. For example, U.S. patent application Publications Nos. 20030212136 and 20030212130 describe several methods for the preparation or creatine esters. See also, for example, Dox et al., Esterification of Creatine, J. Biol. Chem. 1922, 67, 671-673. Additionally, see, for example, U.S. Pat. No. 3,872,099 which describes methods for producing active amino acid esters and U.S. patent application Publication No. 20040030177 further disclosing the preparation of amino acid esters. Other examples are described in U.S. Pat. No. 5,113,009 for the preparation and isolation of a mineral acid salt of an amino acid methyl ester. In this example, a mineral acid is employed to form a salt with the amino group of the amino acid to function as a catalyst for esterification of the carboxylic acid group of the amino acid with an alkyl alcohol, such as methanol. Still other examples of amino acid ester synthesis may be found in EP 1044 676 and EPO 928 608 b y Ajinomoto Co., which is also an excellent source for obtaining pharmaceutical grade amino acids, preferably in crystalline form for use in the present invention.

The preparation of esterified orotate derivatives is also known, such as described, for example, in U.S. patent application Publication No. 20040033981, where an R moiety may be an alcohol containing 1 to 20 carbon atoms joined to orotate via an ester linkage. Additionally, U.S. patent application Publication No. 20040198823 discloses the preparation of creatine orotate, dicreatine orotate and tricreatine orotate, which may be used by themselves in the inventive formulations, or as starting compounds for ester formation, particularly amino acid ester formation and use thereof in the inventive formulations and preparations.

Although these references refer only to some limited examples of esterified creatine and orotic acid compounds, the wide array of esterified creatine and orotic acid products, and esterified creatine-orotate complexes of the present invention, including novel amino acid and/or peptide esterified compounds and compositions, may be produced using analogous reaction systems without departing from the spirit and scope of the present invention

In a preferred embodiment, one or more or a mixture of creatine and/or orotate esters and/or esterified creatine-orotate complexes, or creatine-orotate complexes of the invention as nutrient supplements may be orally administered to or by a user, for example, a person desirous of maintaining energy and promoting muscle and other body tissue development, or to combat fatigue, and to reduce muscle damage during exercise, or to enhance recovery after exercise, or for any end result contemplated. For example, a regimen for enhancing physical performance through amino acid nutritional amino acid supplements as disclosed in U.S. Pat. No. 5,026,721 may be used with the amino acid esters and peptide esters of this invention. The inventive preparations may also be used as pronutrients and/or as anti-inflamatory agents, such as described, for example, in U.S. patent applications Publications Nos. 20030212130 and 20030212136.

In an additional preferred embodiment, for example, the novel compound supplements of the invention may be administered orally individually, or in selected mixtures tailored to achieve a specific end result, for example, such as is known in the art for creatine and orotic acid. Additionally, various and each of the essential and non-essential amino acids, or naturally occurring amino acids and peptides thereof, or oligo-peptides, are known for their individual efficacy. For example, it has been reported that certain amino acids taken as isolated supplements may make up for a deficiency of that amino acid, or may make up for a shortage of chemicals from which an amino acid is synthesized, or for a shortage of molecules or chemicals that are made up from the amino acid, For instance, phenylalanine has been shown to relieve pain syndromes; tryptophan has been shown to raise serotanium levels in the brain to relieve depression; and tyrosine is known to help with depression as well. Glutamine may help balance blood sugar and regenerate the intestinal tract. Further, for example, isoleucine is known for its role with other branched amino acids in the rebuilding of muscle tissue, and in the release of energy, during muscular work, as is leucine. Lysine is known for its promotion of bone growth, and valine is noted for its role in muscle building. Further, for example, as is known, arginine serves as a precursor for the synthesis of nitric oxide, urea, ployamines, proline, glutamine, creatine and agmatine. Endothelium-derived nitric oxide (NO) is a key molecule in the regulation of vascular tone and homeostasis, and its association with vascular disease has long been known. Impaired bioavailability of endothelial NO is associated with hypercholesterolemia and atherosclerosis. Thus, the infusion of L-arginine has been shown to improve endothelium-dependant vasodilation, and to improve endothelial function in, inter alia, atherosclerosis. See, Kurowska, KGK Synergise, Inc., London, Ontario N6A 5R8 Canada. See also, Guoyao, et al., Arginine metabolism: nitric acid and beyond, Biochem. J. (1998) 336, 1-17. Endothelially generated NO has also been reported as essential for maintaining a penile erection. Anderrson et al., Erectile physiological and pathophysiological pathways involved in erectile dysfunction, J. Urol 170 (2 Pt. 2): S6-13. NO is also known to increase blood flow to working muscles (hemodilation) to promote muscle growth, increase strength and speed muscle recovery.

While these are merely some examples of the many efficacious effects of the known amino acids required in manuals, it can be seen that supplementing the supply of amino acids and/or peptides by way of the creatine and/or orotic acid esters, or esterified creatine-orotate complexes of the invention can help improve an assortment of different types of conditions in the body, individually or simultaneously.

In preferred embodiments, the active ingredients for use in the inventive supplements to enhance a mammals body performance, such as physical performance and the like, may comprise, individually, or alone, from about 0.001 grams to about 99.9 grams of the equivalent of creatine and orotic acid, or from about 0.001 weight % to about 80 weight % of a pharmaceutically acceptable carrier material;

As discussed above, It is also contemplated that various amino acid esters of the invention, be they creatine or orotic acid derivatives or esterified creatine-orotate complexes, be provided in peptide form. Peptide synthesis is well known, and specialty peptides may be provided on a made-to-order basis. See for example, Jerini Co. Any of such custom made peptides may be used as starting materials to form an amino-acid ester useful in the present invention.

These examples are merely illustrative of some preferred compounds and their concentration ranges, and any amount of creatine ester or orotate ester, or esterified creatine-orotate complex which displays any efficacy in any amount as a nutrient supplement for humans and animals for any indication, or is perceived to do so, such a placebo, may be employed in the invention.

As stated above, the esterified compounds or complexes of the invention may be used individually or in mixtures, as described above, or in free crystalline form or as pharmaceutically acceptable salts. They may also be used in any form with one or more pharmaceutically acceptable carriers, adjuvants, excipients, or other essential nutrients, such as vitamins, minerals, electrolytes, carbohydrates and the like. The novel compounds and compositions of the invention may be provided as formulations in aqueous solution, such as a liquid drink for oral ingestion, or be formulated as an organic compound, such as in a solid formulation, for instance, granules, tablets, capsules and the like, for oral ingestion, or as a food bar supplement.

As will be appreciated by those skilled in the art, the esterified compounds and compositions of the invention provide for increased absorption and biodelivery of creatine, orotate, amino acids and peptides, by way of their enhanced bioavailability characteristics. Without desiring to limit this invention to theory, it is generally thought that by masking carboxylic acid functional groups of creatine and orotic acid, or of creatine-orotate complexes, through esterification, the efficient delivery of such compounds to the intestine will more readily occur where absorption into the blood stream may occur. The enhanced permeability and solubility characteristics of the inventive compounds are thought to be a function of their increased lipophilicities which allow for better membrane, or cell membrane, permeability, and concomitant enhanced bioavailability. Conversion to biologically active forms of creatine, orotate/orotic acid, amino acids and peptides are then enabled by esterase enzymes present in cells and in the bloodstream.

The phrase “pharmaceutically acceptable carrier” is recognized in the art, and includes a pharmaceutically acceptable material, composition or vehicle, suitable for administering in any way the compound(s) of the present invention to humans or animals. The carriers include, without limitation, liquid or solid filler, diluent, excipient, solvent or encapsulating material, involved in carrying the subject agent and/or compound(s), such as esterified amino acid ester, from one portion of the body, or organ, to another portion of the body, or organ. Each carrier must be “acceptable” in the sense of being compatible with the other ingredients of a formulation, and, of course, not injurious to the patient.

Some examples of materials which can serve as pharmaceutically acceptable carriers include, for instance, sugars, such as lactose, glucose, or sucrose; starches, such as corn starch and potato starch, cellulose and its derivatives, such as carboxymethyl cellulose, ethyl cellulose and cellulose acetate; natural and synthetic water soluble gums, such as powered tragacanth, guar gum and the like; malt; gelatin; talk; excipients, such as cocoa butter and suppository waxes; oils, such as peanut oil, palm oil, cottonseed oil, safflower oil, sesame oil, olive oil, corn oil, palm oil and soybean oil; glycols, such as propylene glycol; polyols, such as glycerine, sorbitol, mannitol and polyethylene glycol; natural and synthetic lipid emulsions, such as Interlipid, esters, such as ethyl oleate and ethyllaurate; agar and other gelling and/or thickening agents; pH adjusting and/or buffering agents, such as magnesium hydroxide, aluminum hydroxide and phosphate buffer solutions; alginic acid and salts thereof; saline; isotonic saline; ethyl alcohol; and other non-toxic compatible substances employed in pharmaceutical formulations.

Other agents and compounds which may be employed include, without limitation, wetting agents, emulsifiers and lubricants, such as sodium lauryl sulfate and magnesium stearate; coloring agents, release agents; coating agents; sweetening, flavoring and fragrance agents; preservatives and antioxidants, such as ascorbic acid, cysteine hydrochloride, sodium bisulfate, sodium meta bisulfite, sodium sulfite and the like. Some non-limiting examples of oil-soluble anti-oxidants useful herein include ascorbal palmitate, butylated hydroxyanisole (BHA), butylated hydroxytoluene (BHT), lecithin, propyl gallate and the like. Also useful herein are one or more metal chelating agents, such as citric acid, ethylenediamine tetraacetic acid (EDTA), sorbitol, tartaric acid, phosphoric acid and the like.

While preferred for oral deployment and ingestion, the compounds, compositions and formulations thereof of this invention include those suitable for nasal, topical, transdermal, buccal, sublingual, rectal, vaginal, pulmonary and/or parenteral administration. Such formulations may be prepared by any conventional method(s) well known in the art of pharmacy, or by any non-conventional method, including convenient preparation by unit dosage form, with some exemplified methods including the step(s) of bring into association one or more compound(s), such as creatine, orotate or amino acid esters or peptides, of the invention with one or more carriers and, optionally, one or more accessory ingredients, preferably uniformly and intimately, and if necessary, or desired, shaping the resulting product, and which will be administered by forms suitable for each administration route, such as by tablet and capsule form, by injection, inhalation, eye lotion, lotion or ointment, and suppository.

By “parenteral administration” and “administered parenterally” as used herein, means modes of administration other than enternal and topical administration, and usually by injection, which includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, aubarachnoid, intraspinal and intrasternal injection and infusion.

The terms “systemic administration” and “administered systemically”, “peripheral administration” and “administered peripherally” as used herein mean the administration of one or more of the inventive compound(s), such as creatine, orotate or amino acid esters, drugs or other material other than directly into the central nervous system, such that it enters a patient's or user's system/body and is subject to metabolism and similar processes, for example, such as subcutaneous administration and oral administration.

Subject to some preferred compounds and their preferred and/or recommended concentration ranges of use as mentioned above, the amount of active ingredient, be it creatine ester, orotate ester, creatine-orotate complex and esters thereof, or amino acid ester and/or peptide ester or other bio-active ingredient, which can be combined with a carrier material to produce a single dosage form will generally be that amount effective to produce a therapeutic effect, or display any amount of efficacy, such as enhanced energy of the user, pain reduction and the like which shall generally referred to herein as a mammal effective nutritional supplement amount. In general, based upon one-hundred percent, this amount will range from less than about 0.0001 percent to more than about 99.999 percent of active ingredient, depending upon such variables such as the nature of the active ingredient employed, the user, the carrier material employed and the end result contemplated. Actual dosage levels of active ingredients in the inventive formulations may be varied so as to obtain an amount of the active ingredient(s) which is effective to achieve the desired end result contemplated or otherwise the desired therapeutic response (effective nutritional supplement amount) for a particular patient or user, composition, and mode of administration.

In another aspect of the invention, there are provided novel methods of conducting one or more business functions comprising the design, production, marketing, distribution, sale, licensing and/or leasing of the inventive compounds, or nutritional supplements formulations thereof and methods of treating and/or administrating same to person's and animals in need or desire thereof. The novel compounds, formulations and methods of the invention provide unique business opportunities heretofore unavailable, and which will enable the capture of a distinct and exclusive market share for its owners and licensees in the important health services sector with the invention's advantages over conventional methodology as described. It is further contemplated that the inventive subject matter herein be employed as a valuable business tool in the generation of business goodwill and as a vehicle for use in conjunction with trade-marks to generate valuable source identificators, as subject matter to form and operate a business entity.

It is to be understood that many modifications and variations besides those preferred embodiments described above may be made in the inventive nutritional compositions of the invention without departing from the scope and spirit of invention and claims. It is also to be understood that the above embodiments are for illustrative purposes only and are not intended to limit the invention and/or the scope of the appended claims in any way.

1. A nutritional and food preparation supplement comprising a compound selected from the formulae: I, II, III, IV, V, VI, VII, VIII, IX and X,
wherein R is a amino acid residue, or peptide or oligo-peptide residue, or is an ester of alcohols and acids comprising one or more of alkyl ester, alkenyl ester, alkynel ester, formates, acetates, acetoacetates, propionates, and esters of higher molecular weight monobosic acids, acrylates, methyl acrylates, esters of hydroxy acids and dibasic acids and esters of aromatic dibasic acids, and wherein said compound is present in a mammal active nutritional supplement amount.

2. The nutritional supplement of claim 1 wherein R is a residue of essential and non-essential amino acids, naturally occurring amino acids and peptides and oligo-peptides thereof, and pharmaceutically acceptable salts thereof.

3. The nutritional supplement of claim 2 wherein R is selected from the group consisting of glycine, alanine, valine, leucine, isoleucine, phenylalanine, proline, serine, threonine, tyrosine, cysteine, cystine, methionine, lysine, arginine, histidine, tryptophan, aspartic acid, glutamic acid, asparagine, glutamine, taurine citrulline and gama butyric acid, and peptides and pharmaceutically acceptable salts thereof.

4. The nutritional supplement of claim 1 wherein said compound is present in amounts ranging from about 0.0001 weight % to about 99.999 weight % of the nutritional supplement.

5. The nutritional supplement of claim 1 which may be administered to mammals by way of one or more selected from the group consisting of oral, nasal, topical, transdermal, buccal, sublingual, rectal, vaginal, pulmonary and parenteral administration.

6. The nutritional supplement of claim 1 wherein said supplement further comprises a pharmaceutically acceptable carrier.

7. The nutritional supplement of claim 6 wherein said compound is present as a pharmaceutically acceptable salt.

8. The nutritional supplement of claim 6 further comprising one or more of additional bio-active compositions, vitamins, minerals, electrolytes and carbohydrates.

9. A method for the production of the supplement of claim 1.

10. A method for the production of the supplement of claim 2.

11. A method for the production of the supplement of claim 3.

12. A method for the production of the supplement of claim 4.

13. A method for the production of the supplement of claim 5.

14. A method for the production of the supplement of claim 6.

15. A method for the production of the supplement of claim 7.

16. A method for the production of the supplement of claim 8.

17. A method for treating humans and/or animals in need thereof comprising and administering the nutritional supplement of claim 1.

18. A method for treating humans and/or animals in need thereof comprising and administering the nutritional supplement of claim 2.

19. A method for treating humans and/or animals in need thereof comprising and administering the nutritional supplement of claim 3.

20. A method for treating humans and/or animals in need thereof comprising and administering the nutritional supplement of claim 4.

21. A method for treating humans and/or animals in need thereof comprising and administering the nutritional supplement of claim 5.

22. A method for treating humans and/or animals in need thereof comprising and administering the nutritional supplement of claim 6.

23. A method for treating humans and/or animals in need thereof comprising and administering the nutritional supplement of claim 7.

24. A method for treating humans and/or animals in need thereof comprising and administering the nutritional supplement of claim 8.

25. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 1.

26. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 2.

27. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 3.

28. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 4.

29. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 5.

30. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 6.

31. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 7.

32. A method of conducting one or more business related functions selected from designing, manufacturing, marketing, using, distributing, selling, licensing, leasing, generating business goodwill, generating trademark rights, and forming and operating a business entity comprising the use of the nutritional supplement of claim 8.
